(19) United States
(12) Patent Application Publication
(10) Pub. No.: US 2018/0125709 A1
(43) Pub. Date: May 10, 2018

(54) DEVICE FOR ADMINISTERING DROPS IN THE EYES

(71) Applicant: Henry Schiffer, Boca Raton, FL (US)

(72) Inventor: Henry Schiffer, Boca Raton, FL (US)

(21) Appl. No.: 15/346,909

(22) Filed: Nov. 9, 2016

(51) Int. Cl. A61F 9/00; G02C 11/00; G02C 7/10

(52) U.S. Cl. CPC A61F 9/0026; A61F 2220/005; G02C 7/104; G02C 11/00

(57) ABSTRACT

A device for assisting the placement of drops in the eyes includes a pair of sunglasses having left and right dark tinted lenses; each of the lenses has an aperture formed therethrough for alignment with the left and right eyes when the sunglasses are worn by the user. An insert is fitted within each aperture with the use of an adhesive or by snap-fit attachment. Each insert includes an annular rim on the exterior surface of the lens surrounding the aperture and providing a seat for receipt of an eye dropper nozzle therein. Each insert may further include a tubular extension that passes through the aperture to approximately the thickness of the lens.

### BACKGROUND OF THE INVENTION

### Field of the Invention

The present invention relates to a device for placing eye drops in the left and right eyes and, more particularly, to sunglasses having lenses with apertures formed therethough that align with the eyes and inserts fitted within the apertures for placing drops from a dropper directly into the eyes when the glasses are worn on the user and the user is looking straight up at approximately 90 degrees from horizontal.

### Discussion of the Related Art

Placing drops in one's eyes with the use of an eye dropper can be a tedious and awkward task. In order to allow the drops to enter the eyes, a person must tilt their head back and look straight up towards the ceiling. Holding the eye dropper directly above the eye, the person must then squeeze the eye dropper so that one or more drops fall into the eye while the eyelids are kept wide open. Unfortunately, the natural reflexes of the eye often cause the eyelids to close while anticipating the drop falling from the dropper and into the eye. When this happens, the process must be repeated which wastes eye drops and causes frustration. Moreover, many people, particularly elderly persons, have difficulty maintaining their hand steady while looking up and holding the eye dropper directly above the eye and squeezing the dropper so that the appropriate amount of drops fall directly into the person's eye. An unsteady or shaky hand can cause the released drop to miss the eye and land on the person's face. This too is frustrating and wastes eye drop medication which may be expensive.

In the past, others have proposed various devices to facilitate the self introduction of eye drops into the eyes. In particular, others have proposed various devices that incorporate the use of eye glasses having clear lenses with various devices fitted to the lenses to assist in alignment of the nozzle or tip of the eye dropper with the user's eyes. The various devices proposed in the related art are rather cumbersome, have a number of moving parts and can be confusing and difficult to use. Moreover, the use of clear lenses allows light to come through the lens which causes glare and distraction to the eyes when attempted to look up towards the dropper during the process of administering drops to the eyes. This glare, caused by the clear lenses allowing light to strike the eyes from angles other than the specific direction of the eye dropper, can cause the user to squint their eyes, thereby making it difficult to land the drops directly into the user's eye without striking the eyelids. Moreover, the light and glare caused by clear lenses in the various devices proposed in the related art make it difficult for the user to focus in the exact direction of the eye dropper nozzle so that the eyes are perfectly aligned with the dropper nozzle and the drops land in the eye rather than on the eyelids or face of the user.

Considering the forgoing, there remains a need in the art for a device that assists with the placement of drops in the eyes of a user, and wherein the device includes a pair of sunglasses having dark tinted lenses and further including inserts fitted to apertures formed through the tinted lenses, and wherein the inserts are specifically adapted to receive the tip of the eye dropper (i.e., the nozzle) so that the eye dropper can be steadily held and squeezed while administering drops to the user's eyes.

### SUMMARY OF THE INVENTION

The present invention is directed to a device for assisting the placement of drops in the eyes includes a pair of sunglasses having left and right dark tinted lenses; each of the lenses has an aperture formed therethrough for alignment with the left and right eyes when the sunglasses are worn by the user. An insert is fitted within each aperture with the use of an adhesive or by snap-fit attachment. Each insert includes an annular rim on the exterior surface of the lens surrounding the aperture and providing a seat for receipt of an eye dropper nozzle therein. Each insert may further include a tubular extension that passes through the aperture to approximately the thickness of the lens.

### OBJECTS AND ADVANTAGES OF THE INVENTION

Considering the forgoing, it is a primary object of the present invention to provide an improved device for administering drops in the eyes, and wherein the device includes a pair of sunglasses having left and right dark tinted lenses and an aperture formed through the lenses for passage of drops therethough, and wherein the dark tinted lenses avoid glare and distraction to the eyes, while allowing the user to focus on the apertures in the dark tinted lenses to properly align the eyes with the eye dropper nozzle.

It is a further object of the present invention to provide an improved device for administering drops in the eyes of a user of the device, and wherein the device includes a pair of sunglasses having left and right dark tinted lenses and apertures formed through the lenses, and further including inserts fitted to the apertures, wherein the inserts are specifically adapted for receipt of the nozzle of the eye dropper therein so that the user can steadily hold the dropper and squeeze drops from the eye dropper without movement of the eye dropper nozzle relative to the user's eyes.

It is still a further object of the present invention to provide an improved device for administering drops in the eyes of a user of the device, and wherein the device includes inserts that are easily glued or snap-fit to the tinted lenses of sunglasses, so that the inserts fit into and/or around apertures in the sunglasses.

It is still a further object of the present invention to provide an improved device for administering drops in the eyes of the user of the device, and wherein the inserts include a flat surface for gluing to the outer surface of the sunglass lenses and a short tubular stub extending therefrom for passage through the aperture formed in the sunglass lenses to thereby properly align and fit the inserts to the sunglass lenses so that a seat and tubular passage of the inserts aligns with the eyes of the user when wearing the sunglasses.

It is still a further object of the present invention to provide an improved device for administering drops in the eyes of the user of the device, and wherein the device is easy to manufacture using existing sunglasses and inserts that are easily glued or snap-fit to the lenses of the sunglasses.

It is yet a further object of the present invention to provide an improved device for administering drops in the eyes of the user of the device, and wherein the device is simple to use, highly effective, and easily affordable.

It is yet a further object of the present invention to provide an improved device for administering drops in the eyes of a user of the device, and wherein the device can be worn as regular sunglasses when not used to administer drops to the eyes.

These and other objects and advantages of the present invention are more readily apparent with reference to the following detailed description taken in conjunction with the accompanying drawings.

### BRIEF DESCRIPTION OF THE DRAWINGS

For a fuller understanding of the nature of the present invention, reference should be made to the following detailed description taken in conjunction with the accompanying drawings in which:

FIG. 1 is a top rear perspective view of the device of the present invention including a pair of sunglasses and inserts fitted to the tinted lenses of the sunglasses, in accordance with one embodiment of the invention;

FIG. 2 is an isolated rear perspective view of an insert of the present invention adapted to be fitted to an aperture formed through the tinted lenses of the sunglasses, in accordance with one embodiment of the present invention;

FIG. 3 is an isolated perspective view showing the insert of FIG. 2 fitted to the exterior side of a sunglass lens of the device of the present invention;

FIG. 4 is a cross-sectional view taken along the plane of the line indicated as 4-4 in FIG. 3;

FIG. 5 is an elevational view showing use of the device of the present invention to administer drops to the eyes of the user;

FIG. 6 is a cross-sectional view showing an alternative embodiment of the insert attached to the exterior surface of a sunglass lens of the device of the present invention, and wherein the insert is aligned with an aperture formed through the sunglass lens;

FIG. 7 is an isolated exploded perspective view showing an annular decal being fitted to the inside surface of the sunglass lens to surround the aperture to provide a clean appearance on the inside of the lenses;

FIG. 8 is a partially exploded top rear perspective view of the device of the present invention showing an insert separated from an aperture in one of the sunglass lens and another insert fitted to the aperture, demonstrating the manner of attachment of the insert to the aperture in each of the lenses; and

FIG. 9 is a front perspective view of the device of the present invention.

Like reference numerals refer to like parts throughout the several views of the drawings.

### DETAILED DESCRIPTION OF THE PREFERRED EMBODIMENTS

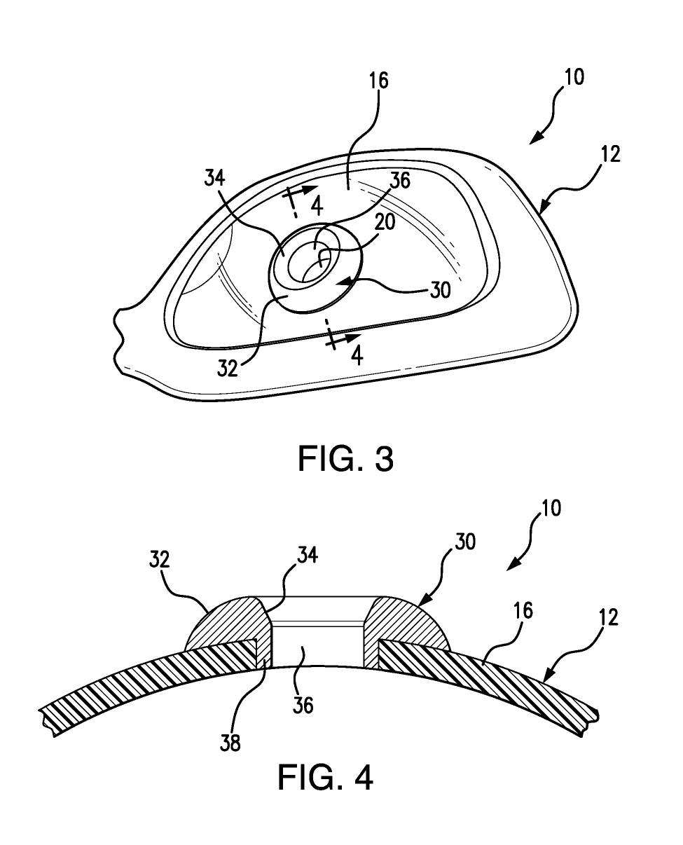

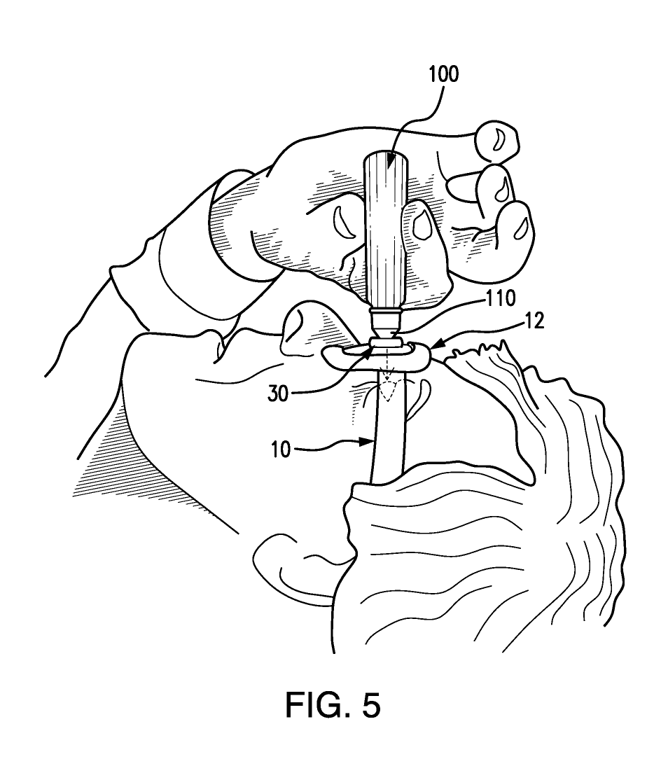

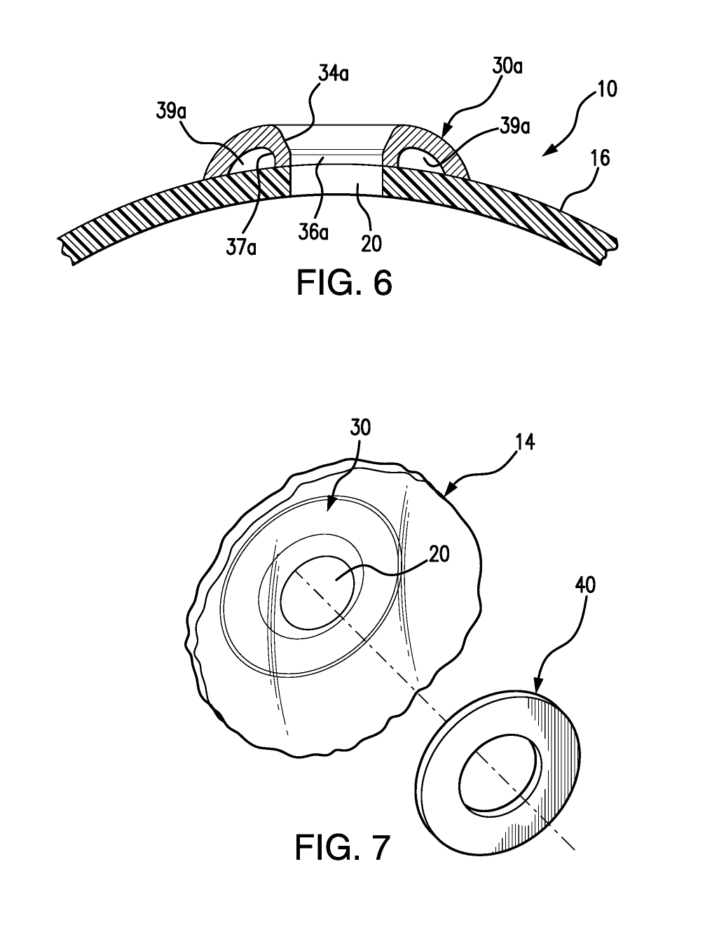

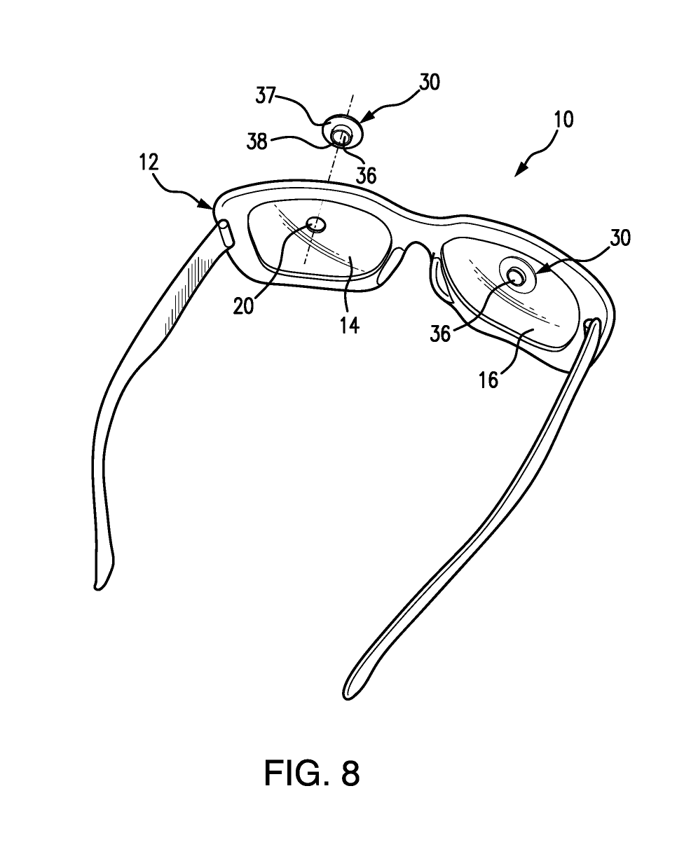

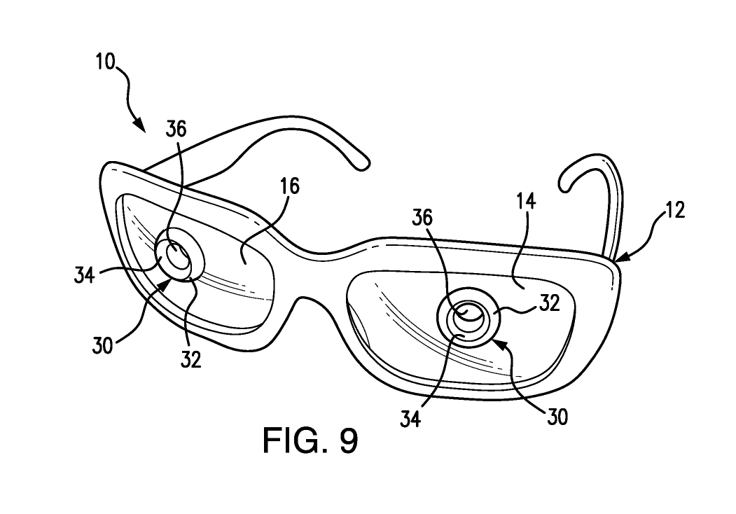

Referring to the several views of the drawings, the present invention is directed to a device 10 for sitting placement of drops in the eyes of the user. The device 10 of the present invention includes a pair of sunglasses 12 having a left dark tinted lens 14 and a right dark tinted lens 16. As seen in FIGS. 4 and 6, each tinted sunglass lens 14, 16 includes an aperture 20 formed therethrough. The aperture 20 in each sunglass lens is specifically positioned to generally align with the user's eyes when the sunglasses are worn in the traditional manner on the user. To assist with proper stabilized positioning of the tip of the nozzle 110 of an eye dropper 100 in alignment with the aperture in each lens, inserts are fitted to each aperture 20. In a preferred embodiment, as seen in FIGS. 2-4, the insert 30 includes an outer annular rim 32 that surrounds a beveled seat 34 and a tubular passage 36. A rear side of the insert preferably includes a flat surface 37 and a short tubular stub 38 extending from the flat surface 37 and surrounding the through passage. The beveled seat is specifically structured for receipt of the nozzle 110 of the eye dropper 100 so that the eye dropper can be held in steady, nonmoving position in direct alignment with the user's eye when the device 10 of the present invention is worn on the user and the user looks directly up towards the sky or ceiling, as seen in FIG. 5. The beveled annular seat in the insert stabilizes the eye dropper so that the eye dropper can be held perfectly still, as seen in FIG. 5, as the user squeezes the eye dropper to administer the proper number of drops to each eye. In this manner, as seen in FIG. 5, the drops are directed straight down and into the eyes of the user, as indicated by the directional arrow. This allows the drops to land directly into the user's eye without landing on the user's eyelids or face. As previously noted, the use of dark tinted sunglasses avoids glare so that users can maintain their eyes wide open, while focusing on the aperture so that the eyes remain in direct alignment with the aperture 20 in each sunglass lens and the drops can then be directed straight down into the user's eye, as shown in FIG. 5.

In the preferred embodiment, the insert 30, as seen in FIGS. 2-4, is adhered to the sunglass lens with an appropriate adhesive. Specifically, an adhesive is applied to the flat surface 37 of the insert 30 and then the insert is placed onto the lens 14, 16, as the tubular stub 38 fits into the aperture 20 of the sunglass lens, as seen in FIG. 4. Alternatively, the short tubular stub 38 can be made to snap-fit into the sunglass lens. In either instance, it is preferable that the length of the tubular stub 38 be no longer than the thickness of the sunglass lens 14, 16, so that the stub 38 does not protrude on the inside of the lens. Ideally, the short tubular stub 38 on the insert 30 should be flush with the inner surface of the sunglass lens 14, 16 when the insert 30 is properly fitted to the lens, as seen in FIG. 4.

In an alternative embodiment, an insert 30a is provided that has a hollow rear side 37a and no tubular stub, as seen in FIG. 6. In this instance, the glue or other adhesive may be applied within the hollow area 39a on the rear side of the insert 30a allowing the insert to be adhered to the outer surface of the sunglass lens 14, 16, with the seat 34a and through passage 36a of the insert 30a properly aligned with the aperture 20 in the sunglass lens 14, 16.

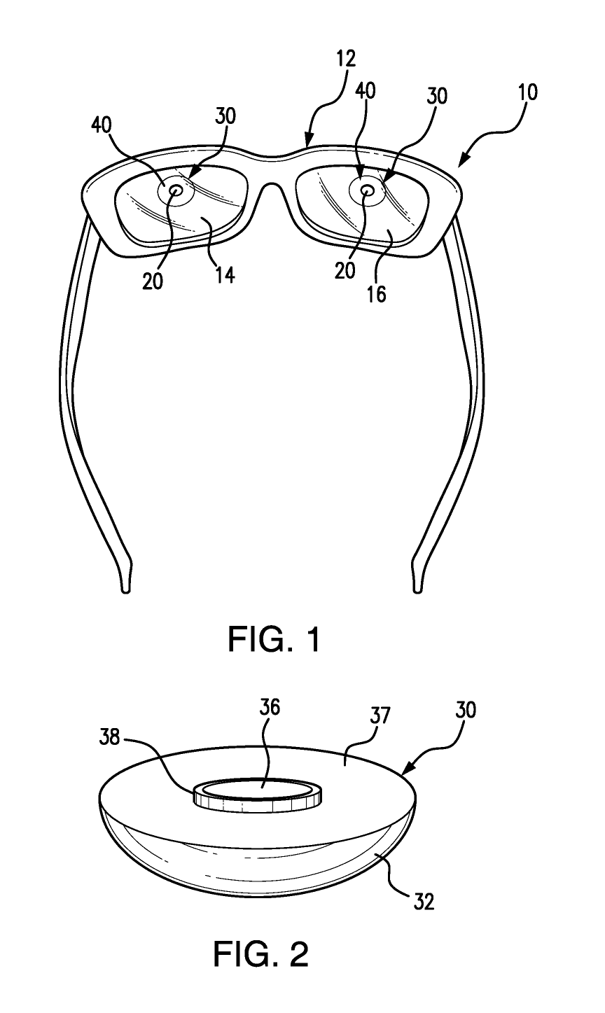

In either embodiment of the invention, a circular or ring shaped decal 40 is attached to the inside surface of each sunglass lens 14, 16, in surrounding relation to the aperture 20 to provide a clean appearance on the inside of the sunglass lens, as seen in FIG. 1. FIG. 7 demonstrates the manner of attachment of the circular decal 40 to the inner surface of the sunglass lens 14, 16 to surround the aperture 20.

What is claimed is:

1. A device for assisting the placement of drops in the eyes of a user, said device comprising:
a pair of sunglasses having left and right dark tinted lenses, and each of the lenses including an aperture formed therethrough for alignment with a respective one of a left and right eye of the user when the sunglasses are worn on the user;
a pair of inserts, including a first insert and a second insert, and each of the inserts being structured and disposed for attachment to an exterior side of a respective one of the left and right dark tinted lenses in aligned relationship with each aperture formed through the left and right dark tinted lenses, and each insert including an annular rim on an exterior side of the lens and surrounding the aperture and providing a seat for receipt of an eye dropper nozzle therein; and
whereby the user, while wearing the sunglasses, and looking straight up at approximately 90 degrees from horizontal, can release drops from the eye dropper nozzle for direct administering into the left and right eyes of the user.

2. The device as recited in claim 1 wherein each insert includes a rear flat surface for mating engagement with the exterior surface of the respective left and right dark tinted lenses in surrounding relationship to the aperture formed through each of the left and right dark tinted lenses.

3. The device as recited in claim 2 wherein the rear flat surface of each insert is attached to the exterior surface of each of the left and right dark tinted lenses with use of an adhesive.

4. The device as recited in claim 2 wherein each of the first and second inserts includes a tubular stub extending from the rear flat surface, and the tubular stub being structured and disposed to pass through the aperture through the respective one of the left and right dark tinted lenses to approximately the thickness of the respective one of the left and right dark tinted lenses.
